SEQUENCE LISTING (2 sequences)

SEQ ID NO: 1 - DNA, 24 bp, mycobacterium avium
tggatggccgaaggagattggccg

SEQ ID NO: 2 - DNA, 24 bp, mycobacterium avium
gttgaggtcgatcgcccacgtgac

(19) United States
(12) Patent Application Publication
(10) Pub. No.: US 2004/0185488 A1
(43) Pub. Date: Sep. 23, 2004

(54) Methods and compositions for diagnosing bovine paratuberculosis

(76) Inventors: Claus D. Buergelt, Gainesville, FL (US); Joseph Elliot Williams, Newberry, FL (US); Jeonghae Park, Gainesville, FL (US)

Correspondence Address: AKERMAN SENTERFITT, P.O. BOX 3188, WEST PALM BEACH, FL, 33402-3188, US

(21) Appl. No.: 10/802,197

(22) Filed: Mar. 17, 2004

Related U.S. Application Data
(60) Provisional application No. 60/455,381, filed on Mar. 17, 2003.

(51) Int. Cl. C12Q 1/68

(52) U.S. Cl. 435/6

(57) ABSTRACT

A method for detecting a Map infection in an animal is performed by subjecting a biological sample isolated from the animal to a nested PCR using at least a first pair of primers for amplifying the ISO900 region of the Map genome and a second pair of primers for amplifying a portion of the amplified ISO900 region. Presence of an amplification product specific for Map in the PCR reaction mixture indicates that the animal is infected with Map. This method is useful for diagnosing both clinical and subclinical paratuberculosis in cows.

### CROSS-REFERENCE TO RELATED APPLICATIONS

The present application claims the priority of U.S. provisional application serial No. 60/455,381 filed Mar. 17, 2003.

### STATEMENT AS TO FEDERALLY SPONSORED RESEARCH

Not applicable.

### FIELD OF THE INVENTION

The invention relates to the fields of microbiology and veterinary medicine. More particularly, the invention relates to polymerase chain reaction (PCR)-based compositions and methods for diagnosing clinical and subclinical bovine paratuberculosis.

### BACKGROUND

Paratuberculosis (Johne's disease) caused by Mycobacterium avium subsp. paratuberculosis (Map), a facultative intracellular, acid-fast bacillus, affects ruminants worldwide. In the United States the disease causes the industry economic losses estimated at $200 and 250 million. The control of the disease is hampered by ineffective diagnostic methods, particularly in detection of infected, subclinical animals.

A fragment of infected animals in a given herd (“iceberg effect”) can be reliably diagnosed based on clinical signs of diarrhea and emaciation, serology, conventional and/or radiometric fecal culture. With infected, subclinical animals, agent detection or serology frequently lead to false negative results. Collins MT. Diagnosis of paratuberculosis. Vet Clin N Am (Food Anim Pract) 1996; 12:357-371. As there are no methods currently available for the management of paratuberculosis such as treatment or vaccination, farmers depend on test and cull programs to control the disease.

Several methods for screening for the presence of Map in tissue samples from affected animals are known. Commonly used immunological methods for detecting Map in a sample include agar gel immunodiffusion (AGID) tests and ELISA assays. More rapid DNA-based tests have been developed that utilize PCR in conjunction with pairs of primers that specifically detect species-specific insertion sequences present in Map strains, but not in other strains of Mycobacterium avium. A commercial DNA-based assay is available for detecting a 413 bp PCR product amplified from the Map insertion sequence defined as IS900 (Vary P H et al., J Clin Microbiol 1990; 28:933-937). While useful, previous PCR-based methods of diagnosing Map infection were not reliably sensitive enough to detect subclinical Map infections. Thus, to better prevent the spread of Map infection in a herd of animals, a more sensitive PCR-based Map detection method would be useful. Methods that allow reliable detection of preclinical infection would be especially desirable.

### SUMMARY

The invention relates to the development of an improved PCR-based method for detecting a subclinical or clinical Map infection in an animal subject. More particularly, the invention utilizes two sets of primers in a “nested PCR” method of detecting Map. In the examples described below, analyses were performed using two pairs of nested PCR primers, the first pair (P90, P91) designed to amplify the 413 bp IS900 sequence, and the second pair (J1, J2) designed to span a 333 bp region within the P90, P91 region. This method was used to detect Map in DNA from samples of blood and milk collected from a large (500 animal) herd of dairy cattle with a proven presence of paratuberculosis. Individuals within the herd population had been followed for several years and were grouped into subclinical categories based on previous ELISA titers for Map. The sensitivity and accuracy of the nested PCR-based method was compared with results from serum ELISA and PCR using a single set of primers on the same samples. Results showed that the nested PCR technique was more sensitive than PCR using the single primer pairs in all four categories of subclinical animals, and that the nested PCR protocol using blood or milk samples was superior to serum ELISA.

Accordingly, the invention features a method for detecting a Map infection in an animal that includes the steps of: (a) providing a biological sample from the animal; and (b) subjecting the biological sample to PCR using primers J1 and J2, wherein the presence of an amplification product specific for Map in the PCR reaction mixture indicates that the animal is infected with Map.

Another method of the invention includes the steps of (A) providing a biological sample from the animal; and (B) subjecting the biological sample to nested PCR using at least a first pair of primers for amplifying the ISO900 region of the Map genome and a second pair of primers for amplifying a portion of the amplified ISO900 region, wherein the presence of an amplification product specific for Map in the PCR reaction mixture indicates that the animal is infected with Map. In this method, the first pair of primers can be primers P90 and P91 and the second pair of primers are primers J1 and J2.

In various aspects of the methods of the invention, the Map infection can be a subclinical infection, the animal can be a cow, and the biological sample can be blood or milk.

In another aspect, the invention features a purified nucleic acid including the nucleotide sequence of SEQ ID NO:1 or SEQ ID NO:2.

Also within the invention is a kit for detecting a Map infection in an animal. The kit can include a first pair of primers for amplifying the ISO900 region of the Map genome and a second pair of primers for amplifying a portion of the amplified ISO900 region. In this kit, the first pair of primers can be primers P90 and P91 and the second pair of primers are primers J1 and J2.

By the term “subclinical” is meant not displaying signs of a disease that are detectable by conventional veterinary or medical examination. In comparison, the term “clinical” means displaying signs of a disease that are detectable by conventional veterinary or medical examination, e.g., rapid weight loss and diarrhea despite good appetite.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control. The particular embodiments discussed below are illustrative only and not intended to be limiting.

### BRIEF DESCRIPTION OF THE DRAWINGS

FIG. 1 is a graph showing ELISA OD readings of cows. Animals #5677 and #5455 maintained fluctuating positive titers (>2.0 OD) after repeated blood collections (up to 13 times). Animals #6142 and #6126 had only 2 blood samples taken, one week apart. Animal #3821 was non-clinical and was used as negative control for ELISA readings.

FIG. 2 is an electrophoretic gel of IS900 amplification products of blood monocytes. Single bands at locations expected for confirmation of Map. Lane 1 DNA extraction from Map lab strain #295 for positive control. Lane 2 positive clinical cows reacting to P90, P91 primers (413bp) on spiked cells. Lane 3 positive clinical cow with reaction products of non-spiked cells. Lanes 4-8 clinical cows (non-spiked cells) reacting to primers J1, J2 (333bp). Lane 9 dH₂O for negative control. Lane 10 molecular size markers (Promega). P=P90, P91 J=J1, J2 M=Molecular Size Markers

FIG. 3 is an electrophoretic gel of IS900 amplification products of milk macrophages. Single bands span over the expected location for second pair of primers J1, J2 (nested PCR). Lane 1 DNA extraction from lab strain #295 for positive control. Lanes 2-3 positive PCR reaction products of spiked cells to P90, P91 at 413bp. Lane 4 reaction product of non-spiked cells. Lane 5 negative control dH₂O. Lane 6 positive PCR reaction product to J1, J2 at 333 bp. Lane 7 molecular size markers (Promega). P=P90, P91 J=J1, J2 M=Molecular Size Markers

FIG. 4 is a graph of ELISA OD readings (2-9 times) from clinical cows that had fluctuating positive OD readings of >2.0.

FIG. 5 is an electrophoretic gel from a group of subclincal cows with negative ELISA OD readings (<1.5), but positive nested PCR IS900 reaction products of non-spiked cells to J1, J2 in 4 milk (M) and 2 blood (B) samples spanning over a 333bp region. Lane 1 positive control; lane 2 #3490M+; lane 3 #3475M+; lane 4 #6091M+; lane 6 #3527M−; lane 7 #3907 M−; lane 8 #3904 M−; lane 9 #3583 M−; lane 10 #3094 M−; lane 11 #3706 M−; lane 12 #6147 M−; lane 13 #3490B+; lane 14 #3379B+; lane 15 dH₂O as negative control; lane 16 molecular size markers (Promega). M=milk B=blood

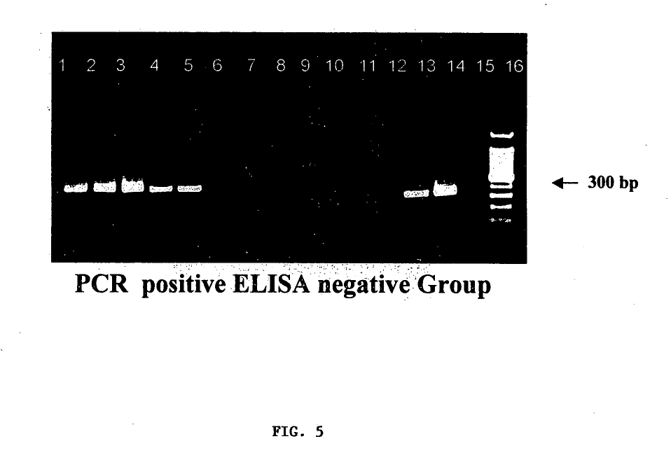

### DETAILED DESCRIPTION

The invention provides methods and compositions for detecting Map infection in an animal. The method includes the steps of providing a biological sample from the animal, and subjecting the biological sample to nested PCR using two sets of primers that result in the amplification of a nucleic acid product specific for Map.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Various techniques using PCR are described, e.g., in Innis et al., PCR Protocols: A Guide to Methods and Applications, Academic Press: San Diego, 1990. Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers.

### Detecting a Map Infection in an Animal

A method for detecting Map infection in an animal includes the steps of first providing a biological sample from the animal, and then subjecting the biological sample to nested PCR using two sets of primers that result in the amplification of a nucleic acid product specific for Map. Any biological sample capable of harboring a Map organism might be used in the invention. The examples below utilize milk and blood. These are preferred as they are readily obtainable from an animal. Other samples from which Map organisms have been isolated include semen, fetuses, spleen, liver and lymph node. Suitable animals from which the sample can be obtained are any that can be infected with a Map organism. A number of such animals are known including ruminants such as sheep and cattle. Because of Map's impact on the commercial bovine milk industry, dairy cows are a particularly preferred source of the biological sample. As the invention is particularly advantageous for diagnosing preclinical Map infections, those animals that do not exhibit clinical signs of paratuberculosis are preferred for use in the invention.

Once isolated, the biological sample can be prepared for PCR analysis according to conventional techniques. For example, the cellular material in blood or milk can be isolated by known methods, e.g., centrifugation through a density gradient. Nucleic acid from the cells can then be purified or partially purified from the collected cellular material by conventional methods, e.g., by boiling in a NaOH solution and centrifugation as described below. To detect the presence of a Map organism, the purified nucleic acid is subjected to nested PCR using primer pairs specific for Map DNA. For detecting low levels of Map in a sample, primer selection is important. For instance, when used in combination with a first primer pair ((P90, P91), the primer pair J1, J2 is advantageous for detecting the low levels of Map that typically occur in subclinically infected cows. Examples of PCR conditions suitable for use in the invention are described in the examples section below, although it is expected that slight modifications to these protocols would still be useful in the invention.

### EXAMPLES

### Example 1

Materials and Methods

Source for sample acquisition and study design. Blood and milk samples were collected from Holstein dairy cows belonging to a 500 animal herd near Gainesville, Fla. The herd had a proven presence of paratuberculosis and was surveyed for three years via ELISA, AGID and necropsy of clinical cases. Before starting this project, a total of 860 serum samples were tested serologically and 18 cases were submitted to necropsy to confirm the clinical diagnosis of paratuberculosis. Eleven clinical cases of paratuberculosis were selected for the purpose of standardizing the chosen PCR method. The cows were in lactation and ranged in age between 4 and 7 years. After verification of the repeatability of the procedure and streamlining individual procedural steps, 4 subsequent study groups were constructed for the testing of subclinical animals. These study groups included animals with a previous negative ELISA status (<1.5 OD), animals with previous suspicious ELISA status (1.5-1.9 OD); animals with an originally low positive ELISA status (2.0-2.5 OD) and animals; with a good (high) positive ELISA status (>2.5 OD). Each of the four study groups was composed of at least 10 animals. A total of 57 animals were tested.

Sample handling. Each cow had 10 ml of blood drawn off the coccygeal vein into vacutainer® tubes containing EDTA, and 50 ml of raw milk drawn off one randomly chosen quarter by hand milking into a sterile centrifuge tube. Three ml of whole blood was added to 4 ml of Ficoll-Isopaque™ plus gradient (Amersham Pharmacia, density 1.078 g/ml) and centrifuged for 30-40 minutes at 500×g at 18° C. Supernatant was removed, and cells were washed with 2 cycles of PBS (NaCl 43.3 gm, Na₂HPO₄ 11.4 gm, KH₂PO₄ 1.33 gm, pH 7.3) and centrifuged at 500×g for 15 minutes. Cells were counted with a haemocytometer, resuspended in 100 ul of 0.2N NaOH, boiled at 110° C. for 20 minutes to extract DNA and re-centrifuged at 500×g for 3 minutes.

Milk samples collected in 50 ml sterile centrifuge tubes were centrifuged at 1,000×g for 15 minutes. Following centrifugation the supernatant was removed and discarded. The samples were washed 3× in PBS. The supernatant was collected and discarded. The remaining sample was suspended in 1 ml PBS for counting, re-suspended in 100 ul of 0.2 N NaOH and boiled as described above for blood.

Blood and milk samples from the first two clinical animals (#5677, #5455) were separated into 2 groups: cells spiked with the laboratory strain #295 of Map and non-spiked cells. The spiked cells were treated with 100 ul of 10⁸ Map for 4 hours at 37° C. in 5% CO₂. For the PCR procedures performed on all remaining animals, only non-spiked cells were used.

Polymerase Chain Reaction. After DNA extraction, lysates and on occasion cellular debris were submitted for PCR. A commercial reaction mix (Fail Safe, EPICENTRE, Wisconsin) was used according to the company's specification. PCR protocol was 35 cycles of 30 sec at 94° C., at 58° C. for 15 sec and at 72° C. for 60 sec. A volume of 10 ul of the PCR was run on 1.5% agarose gel by electrophoresis in TAE running buffer (Continental Lab Products, CA) by current standards. Gel inspection was done using ultraviolet light, and a computerized digital camera was used to take photographs (UVP Transilluminator System).

Samples were probed with primers P90, P91 for IS900 which specifically recognize a 413 bp sequence of Map. See, Vary et al., Journal of Clinical Microbiology 28:933-937,1990. With the exception of the first two clinical cases a nested PCR procedure was performed with a second set of primers designated as J1 (TGGATGGCCGAAGGAGATTGGCCG) (SEQ ID NO:1) and J2 (GTTGAGGTCGATCGCCCACGTGAC) (SEQ ID NO:2). Primer exactness was checked with the lab strain #295 using the two sets of primers. PCR products were sequenced for nucleotide homology using GenBank as database. Homologies of 99.9% and 100% were obtained respectively for P90, P91 and J1, J2. DNA extracted from the lab strain #295 was used as positive control and dH₂O as negative control. Additional primer exactness was tested by submitting original samples to a set of P1, P2 primers, recognizing a 427 bp sequence (IS1245) of Mycobacterium avium subsp. avium (Maa) and a third set of primers, DD2, DD3, probing for the insertion sequence IS1311 which identifies a 180 bp sequence shared by Map and Maa.

ELISA. The enzyme-linked immunosorbent assay was performed with a crude, soluble protoplasmic antigen (Allied Monitor, Missouri). Test sera were pre-absorbed with Mycobacterium phlei. ELISA results were computerized from wavelength readings (OD at 405 nm) of triplicates and recorded as negative (<1.5 OD), suspicious (1.5-1.9 OD), low positive (2.0-2.5 OD) and high positive (>2.5 OD).

AGID. For the agar-gel immunodiffusion test the same crude, protoplasmatic antigen as for the ELISA was used. Readings were done after 48 hours.

Necropsy and histopathology. Animals sacrificed were submitted for a complete necropsy. Major organs were sampled for histopathology including small intestine and mesenteric lymph nodes. Sections were stained with hematoxylin-eosin (H&E) and acid-fast stains (Fite's) before examination under a light microscope.

Statistical Analyses. Significance was determined by McNemar's test (Paired Chi-square). A probability value of 0.05 was considered to be statistically significant.

Results

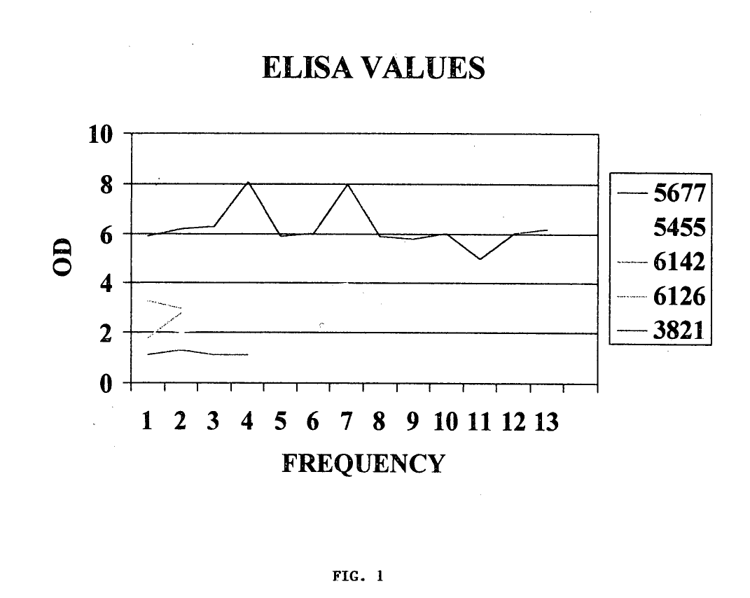

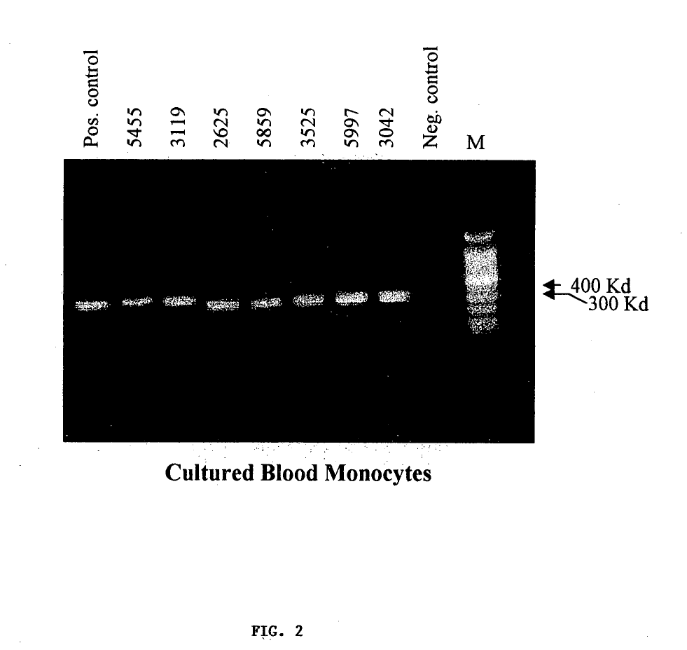

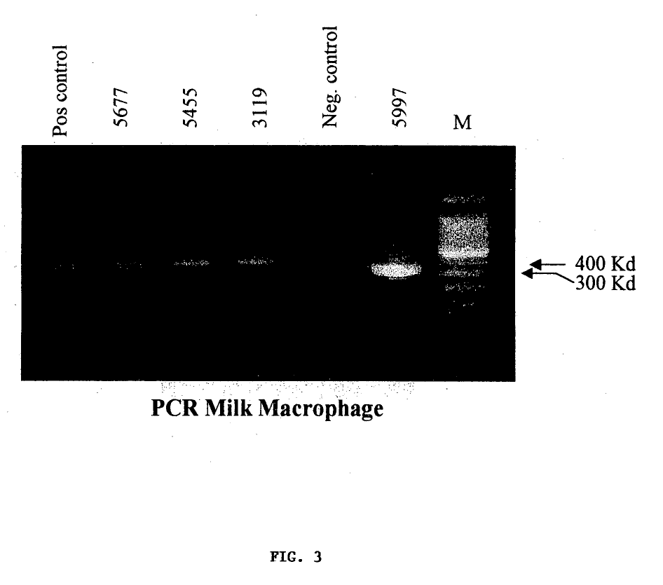

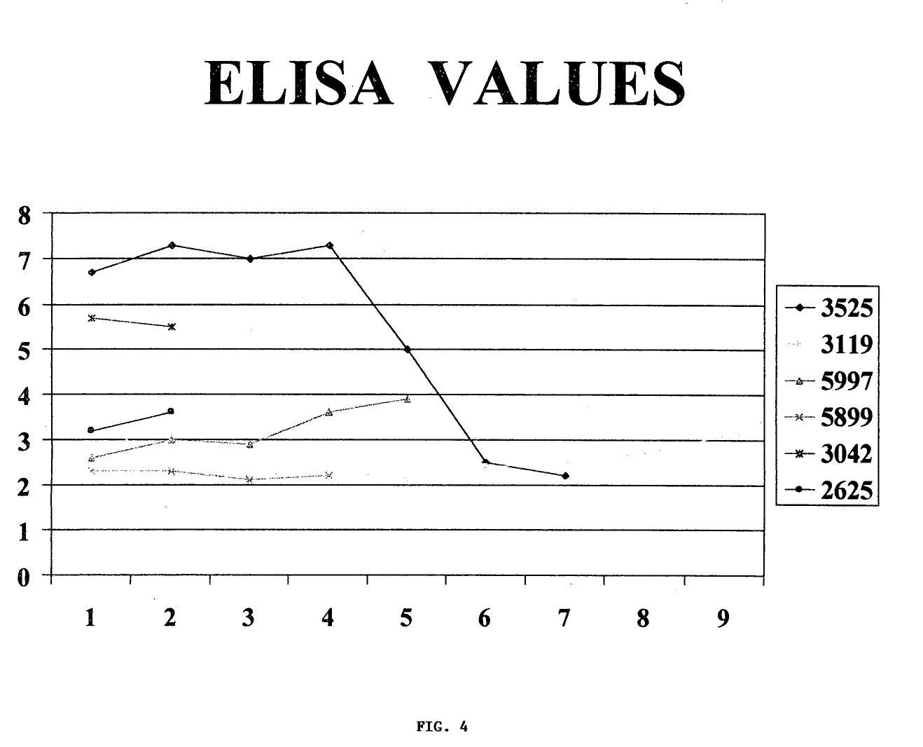

The group of cows showing clinical signs characterized by weight loss and diarrhea despite good appetite was composed of 11 cows ranging in age between 4-7 years (mean 5.5 years). All but one was positive on ELISA; 6 cows were positive on AGID. The first two animals (#5677, #5455) from this group were used to manipulate and to standardize the PCR system. The animals were repeatedly bled and milked, an average of twice weekly for 3 weeks. Blood and milk samples when subjected to primer sets P90, P91; DD3, DD4; P1, P2 gave PCR products (amplicons) specifically positive for Map when probed with P90, P91 and with DD3, DD4, and no PCR reaction products when probed with primers P1, P2. Presence of single bands for the expected base pairs showed that blood monocytes and milk macrophages contained Map. Repeated ELISA tests (FIG. 1) showed that both cohorts maintained a positive titer above the diagnostic level of 2.0 for Johne's disease despite a varying pattern (frequency of 10 or more OD readings). The remaining 9 cows were subjected to a nested PCR with 2 sets of primers (P90, P91 and J1, J2). During these experiments only one cow reacted positively to the set of P90, P91 primers while the others showed PCR products on blood, milk or both with the second set of primers J1, J2. (Table 1). A comparison of results between the serologic tests and the PCR procedures is listed in Table 6. Representative PCR blood and milk PCR products are shown in FIGS. 2 and 3. All animals maintained a positive ELISA (FIG. 4) profile (frequency of 2-9 bleedings). While the milk samples from 10 animals were collected from one randomly chosen quarter, cow #6142 was tested on all 4 quarters separately. Three of the quarters were positive on PCR, while one was negative. The cow was retested 3 months later with only one quarter being positive on PCR. A positive signal also was obtained with a pooled sample from all 4 quarters at that time. A third testing 4 weeks later had 3 of the 4 quarters reacting positively and also the pooled milk sample from all 4 quarters. The animal reacted on the nested blood PCR only at the third testing.

The study group of the subclinical cases selected from ELISA values originally obtained a year ago gave results summarized in Tables 2-5. The group of cows (N=11) with high ELISA readings, aged between 3 and 7.5 years (mean of 5.1 years) maintained, dropped or was negative on ELISA by the time the blood and milk samples were collected for PCR. All animals were negative on AGID. All 11 animals had a positive PCR reaction product on nested PCR; 9 cows, showed amplicons in both blood and milk samples; one each in milk and blood only. Five of the animals reacted with the first set of primers P90, P91. as well. (Table 2).

The subclinical group with an original low ELISA value consisted of 11 animals ranging in age from 2.5-9.5 years (4.7 years average). All but one gave suspicious to low positive ELISA values when tested a second time a year later. All were negative on AGID. All were negative when subjected to the set of primers P90, P91; 4 cows had positive amplicons on milk, blood or both when probed with the second-set of J1, J2. (Table 3).

Of the animals from the subclinical group (N=11) chosen because of their original suspicious ELISA value had four cows reacting positively with the nested PCR (Table 4). The animals ranged in age between 3-8.5 years (4.0 years mean). Only one had maintained a suspicious ELISA value; all were negative on AGID.

The study group of subclinical animals (N=12) originally negative on ELISA (N=8) had negative ELISA values when repeated. Four animals not originally analyzed serologically were added to the group. The animals ranged in age between 3 and 11 years (mean 5.0 years). All animals were negative on AGID. From this group five animals had positive nested PCR reaction products on milk, blood or both (Table 5). A comparison between the serologic results and the nested PCR procedures of animals belonging to the four subclinical groups are listed in Table 7.

Comparison of test sensitivity between ELISA and PCR using McNemar's test had a chi-square distribution x² of 16.0 and a p value of <0.0001 indicating a significant difference in sensitivity between the two tests in the cohorts of subclinical animals. Agreement between ELISA and PCR tests is summarized in Table 8.

TABLE 1
Summary of Results of Clinical Cases of Paratuberculosis. (N = 11)

| Cow # | Age (Years) | AGID | ELISA | P90, P91 | J1, J2 |
|---|---|---|---|---|---|
| 5677 | 5 | + | + | M | ND |
| 5455 | 6 | + | + | B, M | ND |
| 5859 | 7 | − | + | — | B |
| 3119 | 5 | + | + | B(d), M | M |
| 2625 | 4 | + | + | — | B, S |
| 3042 | 6 | − | + | — | B, M |
| 5997 | 6 | − | + | — | B, M |
| 3525 | 4 | + | − | — | B |
| 6142 | 5 | + | + | — | M (3x) |
| 6126 | 5 | − | + | — | M |
| 2989 | 7 | − | + | — | B |

B = blood
M = milk 3x sampling
S = spleen
d = cellular debris
ND = not done
P90, P91 1st set of primers
J1, J2 2nd set of primers

TABLE 2
Summary of Results of Subclinical Group with Good ELISA (N = 11)

| Cow # | Age (Years) | AGID | ELISA | P90, P91 | J1, J2 |
|---|---|---|---|---|---|
| 5659 | 7 | − | 2.7 (3.0) | — | B, M |
| 5801 | 7.5 | − | 2.8 (2.8) | — | M |
| 5947 | 6 | − | 1.8 (2.8) | M | M |
| 3362 | 5 | − | 2.0 (3.8) | B | B, M |
| 3873 | 3 | − | —(2.9) | — | B, M |
| 3907 | 3 | − | —(3.8) | — | B, M |
| 3190 | 5.5 | − | —(2.5) | M | B, M |
| 3527 | ND | − | —(2.5) | M | B, M |
| 5801 | 7 | − | 1.7 (2.8) | — | B, M |
| 3494 | 4.5 | − | —(2.5) | — | B, M |
| 3900 | 3 | − | 3.1 (ND) | B | B |

() Value of 1st ELISA

TABLE 3
Summary of Results of Subclinical
Group with Low Positive ELISA. (N = 11)

| Cow # | Age (Years) | AGID | ELISA | P90, P91 | J1, J2 |
|---|---|---|---|---|---|
| 3584 | 4 | − | 2.3 (2.4) | — | — |
| 3418 | 5 | − | 2.1 (2.3) | — | — |
| 3820 | 3 | − | 1.7 (2.3) | — | — |
| 5560 | 9.5 | − | 2.3 (2.3) | — | M |
| 3973 | 3 | − | 1.6 (2.1) | — | M |
| 3858 | 4 | − | 3.6 (2.2) | — | B, M |
| 3976 | 3 | − | 2.2 (2.1) | — | B, M |
| 3941 | 3 | − | 1.8 (2.3) | — | — |
| 4151 | 2.5 | − | —(2.1) | — | — |
| 6130 | 5 | − | 1.9 (2.0) | — | — |
| 6093 | 6.5 | − | 2.3 (2.0) | — | — |

TABLE 4
Summary of Results of Subclinical Group
with Suspicious ELISA. (N = 11)

| Cow # | Age (Years) | AGID | ELISA | P90, P91 | J1, J2 |
|---|---|---|---|---|---|
| 3557 | 4 | − | 1.9 (1.7) | — | — |
| 3849 | 3 | − | —(1.7) | — | — |
| 3925 | 3 | − | 1.8 (1.8) | — | — |
| 3824 | 3 | − | —(1.9) | — | — |
| 3794 | 4 | − | —(1.9) | — | M |
| 3814 | 4 | − | —(1.9) | — | B, M |
| 2854 | 8.5 | − | —(1.8) | — | — |
| 3944 | 3 | − | —(1.9) | — | — |
| 3926 | 3 | − | —(1.9) | — | M |
| 3946 | 3 | − | —(1.7) | — | — |
| 6028 | 6 | − | —(1.5) | — | B, M |

TABLE 5
Summary of Results of Subclinical
Group with Negative ELISA. (N = 12)

| Cow # | Age (Years) | AGID | ELISA | P90, P91 | J1, J2 |
|---|---|---|---|---|---|
| 3490 | 5.5 | − | —(−) | — | B, M |
| 3475 | 4.5 | − | —(−) | — | M |
| 6091 | 11 | − | —(ND) | — | M |
| 6044 | 5.5 | − | —(ND) | — | M |
| 3527 | 4.5 | − | —(−) | — | — |
| 3907 | 3 | − | —(−) | — | — |
| 3904 | 3 | − | —(−) | — | — |
| 3379 | 3 | − | —(−) | — | B |
| 3583 | 4.5 | − | —(−) | — | — |
| 3094 | 6 | − | —(−) | — | — |
| 3706 | 4.5 | − | —(ND) | — | — |
| 6147 | 5 | − | —(ND) | — | — |

TABLE 6
Summary of Results of Clinical Cases. (N = 11)

| | |
|---|---|
| Positive on Blood PCR*: | 4 |
| Positive on Milk PCR*: | 2 |
| Positive on Milk and Blood PCR*: | 4 |
| Total PCR Positive: | 11 |
| Positive on ELISA: | 10 |
| Positive on AGID: | 6 |

*Combined single and nested

TABLE 7
Summary of Results of Sublinical Groups. (N = 45)

| | |
|---|---|
| Positive on Blood nPCR: | 2 |
| Positive on Milk nPCR: | 9 |
| Positive on Blood and Milk nPCR: | 13 |
| Total nPCR Positive: | 24 |
| Positive and Suspicious ELISA: | 18 |
| Positive on AGID: | 0 |

TABLE 8
ELISA and Nested PCR Agreement of Subclinical Groups Cows.

| | NPCR Positive | nPCR Negative | Total |
|---|---|---|---|
| ELISA Pos/Susp. | 10(22%) | 8(18%) | 18 |
| ELISA Negative | 14(31%) | 13(29%) | 27 |
| Total | 24 | 21 | 45 |

### Example 2

The nested PCR methods described in Example 1 have also successfully been used to detect Map in bull semen of bulls and fetal fluids and fetal tissues of pregnant cows.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting a Map infection in an animal, the method comprising the steps of:
(A) providing a biological sample from the animal; and
(B) subjecting the biological sample to PCR using primers J1 and J2, wherein the presence of an amplification product specific for Map in the PCR reaction mixture indicates that the animal is infected with Map.

2. The method of claim 1, wherein the Map infection is a subclinical infection.

3. The method of claim 1, wherein the animal is a cow.

4. The method of claim 1, wherein the biological sample is blood.

5. The method of claim 1, wherein the biological sample is milk.

6. A method for detecting a Map infection in an animal, the method comprising the steps of:
(A) providing a biological sample from the animal; and
(B) subjecting the biological sample to nested PCR using at least a first pair of primers for amplifying the ISO900 region of the Map genome and a second pair of primers for amplifying a portion of the amplified ISO900 region,
wherein the presence of an amplification product specific for Map in the PCR reaction mixture indicates that the animal is infected with Map.

7. The method of claim 6, wherein the first pair of primers are primers P90 and P91.

8. The method of claim 6, wherein the second pair of primers are primers J1 and J2.

9. The method of claim 7, wherein the second pair of primers are primers J1 and J2.

10. The method of claim 6, wherein the Map infection is a subclinical infection.

11. The method of claim 6, wherein the animal is a cow.

12. The method of claim 6, wherein the biological sample is blood.

13. The method of claim 6, wherein the biological sample is milk.

14. The method of claim 6, wherein the first set of primers consist of the primers P90 and P91.

15. A purified nucleic acid comprising the nucleotide sequence of SEQ ID NO:1.

16. A purified nucleic acid comprising the nucleotide sequence of SEQ ID NO:2.

17. A kit for detecting a Map infection in an animal, the kit comprising a first pair, of primers for amplifying the ISO900 region of the Map genome and a second pair of primers for amplifying a portion of the amplified ISO900region.

18. The kit of claim 17, wherein the first pair, of primers are primers P90 and P91.

19. The kit of claim 17, wherein the second pair of primers are primers J1 and J2.

20. The kit of claim 18, wherein the second pair of primers are primers J1 and J2.
